(19) United States
(12) Patent Application Publication
(10) Pub. No.: US 2005/0069865 A1
(43) Pub. Date: Mar. 31, 2005

(54) Synthetic hcv envelope proteins and their use for vaccination

(76) Inventors: Lewis Neville, Rehovot (IL); Arie Zauberman, Nes-Ziona (IL)

Correspondence Address: HELLER EHRMAN WHITE & MCAULIFFE LLP, 1666 K STREET,NW, SUITE 300, WASHINGTON, DC, 20006, US

(21) Appl. No.: 10/488,269

(22) PCT Filed: Sep. 9, 2002
(86) PCT No.: PCT/IL02/00744

(30) Foreign Application Priority Data
Sep. 13, 2001 (IL) 145440

(51) Int. Cl. C12Q 1/70; C07H 21/04; A61K 39/29; C12N 7/00; C07K 14/02

(52) U.S. Cl. 435/5; 424/189.1; 435/69.3; 435/235.1; 435/325; 435/456; 530/350; 536/23.53

(57) ABSTRACT

The present invention relates to the amino acid and deduced nucleic acid sequence of synthetic E2 proteins comprising a consensus sequence of the most conserved amino acids found in E2 of 77 different HCV 1b isolates worldwide. The invention further relates to a truncated version of the E2 protein lacking HVR1. Another aspect of the present invention concerns a vaccine comprising the E2 proteins.

### FIELD OF THE INVENTION

The present invention is in the field of hepatitis virology. The invention relates to the amino acid sequence of rationally designed synthetic E2 proteins comprising a consensus sequence of the most conserved amino acids found in E2 of 77 different HCV 1b isolates from around the world. More specifically, this invention relates to a vaccine comprising the synthetic E2 proteins.

### BACKGROUND OF THE INVENTION

Hepatitis C virus (HCV) is a single stranded positive RNA virus that has been classified as a member of the Flaviviridae genus (Bartenschlager and Lohmann, 2000 J. Gen. Virology; 81 Pt 7:1631-48). Its genome consists of a highly conserved 5′ non-coding region followed by a single open reading frame of approximately 10,000 nucleotides that is translated as a polyprotein precursor of 3010-3033 amino acids. Subsequent enzymatic cleavage via host and HCV-encoded proteases (Hijikata et al, 1991 PNAS USA 88, 5547-51; Lin et al, 1994 J Virology 68, 5063-73; Lin et al, 1994 J Virology 68, 8147-57 Grakoui et al, 1993 PNAS USA 90, 10583-7; Grakoui et al, 1993 J. Virology 67, 2832-43) yields at least 10 different polypeptides which can be divided into structural: Core and Envelope proteins and non-structural proteins: helicase, protease, RNA-dependent RNA polymerase. Thereby constituting the HCV virion (Miyamura and Matsuura, 1993 Trends Microbiology 1(6): 229-31; Bartenschlager and Lohmann, 2000 J. Gen. Virology; 81 Pt 7:1631-48). HCV is a major public health concern due to its ability to generate a relentless infection that results in chronic liver disease and in some cases, hepatocellular carcinoma (Hoofnagel, 1997 Hepatology 26 (3 Suppl 1):15S-20S). At present, anti-viral therapy is insufficient and the development of improved therapeutics and an effective HCV vaccine is therefore of high priority (for review see Rosen and Gretch, 1999 Mol Med Today 5(9): 393-9).

The development of efficacious anti-HCV drugs including an HCV vaccine is thwarted by the fact that HCV exists as a panel of at least six different proteins derived from genotypes 1-6 (Simmonds et al, 1993 J. of Gen. Virology 74, 2391-9). To add further complexity, an individual infected with HCV is likely to contain a spectrum of different HCV quasi-species derived from one dominant genotype (Forms et al, 1999 Trends Microbiology 7(10): 402-10). This intrinsic hyper-mutability of HCV is caused by the low fidelity of HCV RNA Polymerase (NS5B; Bartenschlager and Lohmann, 2000 J. Gen. Virology; 81 Pt 7:1631-48). Consequently, certain HCV peptide motifs that are presumably hydrophilic in nature and surface-expressed, are immunogenic but highly mutable, and can escape from immune surveillance. One motif which conforms to these properties has been coined the hyper-variable region I (HVR1) and constitutes a 27 amino acid stretch at the N′-terminus of the envelope protein E2. The HVR1 contains a number of T and B cell epitopes (Weiner et al, 1992 PNAS USA 89(8): 3468-72; Scarselli et al, 1995 J Virology 69(7): 4407-12; Zibert et al, 1995 Virology 208, 653-61) and antibodies against this domain have been shown to inhibit binding and infection of HCV to human fibroblast cells (Zibert et al, 1995 Virology 208, 653-61; Shimizu et al, 1996 Virology 223(2): 409-12), to partially abrogate E2 CHO binding to MOLT-4 cells in a neutralization of binding (NOB) assay (Rosa et al, 1996 PNAS USA 93, 1759-63), to capture HCV in immuno-precipitation assays (Esumi et al, 1996 J Virol Methods 59 (1-2): 91-8) and to ameliorate at least in part, HCV infectivity in chimpanzees (Farci et al, 1994 PNAS USA 91(16):7792-6; Farci et al, 1996 PNAS USA 93, 15394-9; Shimuzu et al, 1996 Virology 223(2): 409-12).

Many efforts were dedicated to studying the HVR1 as a vaccine candidate, for example, Goto et al., (2001 Hepatology Research 19:270-283) immunized a chimpanzee with synthetic HVR1 peptides and achieved protection. Carlos et al., (2000, Vaccine Weekly, July 26 p.17) report designing a synthetic construct that incorporates a number of the mutations generally found in the hypervariable regions of the virus. This peptide construct included material found in HVR1 and HVR2 of the virus.

Additionally important domains involved in HCV neutralization are thought to reside outside the HVR1 since a protective vaccine comprising E1/E2 generated a hyperimmune serum with extremely low binding titers to HVR1 (Choo et al., 1994 PNAS USA 90: 1294-1298). Choo's vaccine however was protective only against a homologous strain of HCV.

Several attempts have been made to use the E2 protein for immunization. Bukh et al in WO 200121807 disclose a nucleic acid molecule encoding HCV lacking HVR1 and its use for immunization. Zucchelli et al., (2001 Hepatology 33: 692-703) disclose certain HVR1 peptide mimics (mimotopes) fused to the ectodomain of the E2 protein that were able to induce a strong humoral response.

It seems therefore that the E2 envelope protein as a whole represents a feasible target site to neutralize HCV infection. Hence, the development of an appropriate E2 antigen as a prophylactic as well as therapeutic vaccine suitable for heterologous HCV subtypes is of the utmost importance.

### SUMMARY OF THE INVENTION

The present invention relates to the amino acid and deduced nucleic acid sequences of a synthetic E2 protein comprising a consensus sequence of the most conserved amino acids found in E2 of 77 different HCV 1b isolates (“E2 majority”). The invention also relates to the amino and deduced nucleotide sequences of a truncated E2 protein lacking HVR1 (“E2 majority w/o”) and to the amino and deduced nucleotide sequences of an E2 majority protein wherein the HVR1 was replaced with the R9 mimotope (Puntoriero et al., The EMBO Journal Vol. 17, No. 13 pp3521-3533, 1998) (“E2 majority R9”). The invention also relates to variants of the E2 majority proteins of the invention having at least 98% homology to the disclosed sequences.

The invention further relates to proteins derived from the sequences disclosed herein.

These proteins may be produced by recombinant methods by inserting the nucleic acid sequences encoding the E2 proteins of the invention into an expression vector and expressing the recombinant proteins in a host cell.

One aspect of the invention relates to the use of these proteins as vaccines.

Another aspect of the invention relates to the use of expression vectors containing the nucleic acid sequences encoding the E2 proteins of the present invention as nucleic acid based vaccines.

This invention further relates to pharmaceutical compositions comprising the proteins of the invention for use in prevention or treatment of hepatitis C in an individual.

The proteins of the present invention can also be used for detecting antibodies specific for HCV in biological samples. And therefore can serve as diagnostic tools to identify and monitor HCV infection, disease progression and efficacy of therapeutic agents during the course of treatment of HCV infection.

Another aspect of the present invention is a kit for the detection of antibodies specific for HCV in a biological sample wherein said kit comprises essentially a purified and isolated protein of the invention.

Another aspect of the invention relates to antibodies to the E2 proteins of the present invention and to the use of such antibodies in passive immunotherapy or prophylaxis.

### BRIEF DESCRIPTION OF THE DRAWINGS

FIG. 1. Amino acid sequences of E2 majority, E2 majority R9 and E2 majority-w/o. Putative glycosylation sites are underlined.

FIG. 2. A photograph of a Western blot showing reactivity of different HCV sera genotypes to various E2 proteins. Purified baculovirus-produced, HCV E2 proteins (lane nos. 1-4), a mammalian Chinese hamster ovary (CHO) cell produced E2 (lane no. 5), and a non relevant negative control protein thioredoxin (lane no. 6) were run on SDS-PAGE gels under non-reducing conditions and probed with different human sera representing HCV genotypes 1a, 1b, 2a/2c, 3a, 4, 5 and 6. Controls included the following: an anti-histidine monoclonal antibody (α-His mAb), AB^{XTL}68 a human anti-E2 mAb, served as a positive control, anti-HCV negative (αHCV-ve) and anti-Hepatitis B Core positive (αHBC+ve) sera were included as negative controls.

FIG. 3. A photograph of a Western blot showing reactivity of different HCV sera genotypes to various E2 proteins. Purified baculovirus-produced, natural E2 and E2 majority proteins (lane nos. 1 and 2), and a non relevant negative control protein thioredoxin (lane no. 3) were run on SDS-PAGE gels under non-reducing conditions and probed with different human sera representing HCV genotypes 1b, 2a/2c, 3a, and 4. AB^{XTL}68 a human anti-E2 mAb, served as a positive control.

FIG. 4. is a graphic representation of the binding properties of hyperimmune sera generated by immunization of naïve mice with various E2 proteins. In (A) the binding was examined using ELISA plates coated with a variety of antigens. Each box represents a different antigen used for coating. Data are presented as O.D. measurements as a function of serum dilution. In (B) binding was examined using a Western blot wherein various E2 preparations were run on the gel and reacted with different hyperimmune sera.

FIG. 5. is a graphic representation of the binding properties of mouse monoclonal antibodies to various E2 preparations and to thioredoxin, which serves as a negative control. mAb18 was raised against natural E2 and mAb 21 was raised against E2 majority R9. Binding was examined using a Western blot wherein various E2 preparations were run on the gel and reacted with the different monoclonal antibodies. Numbers on the left side of the blot represent the estimated molecular weight in kD.

FIG. 6. is a graphic representation of the mean viral load and percentage of HCV-Trimera mice with positive HCV RT-PCR signal in their serum (numbers in parentheses) at day 19 after transplantation. The bars represent different experimental groups: a group wherein the transplanted liver was pre-incubated with HCV infectious serum and a pre-immune serum (control); and groups wherein the transplanted liver was pre-incubated with HCV infectious serum and various anti E2 IgG preparations.

Reference will now be made to the following examples that are provided by way of illustration and are not intended to be limiting to the present invention.

### EXAMPLES

The invention relates to the surprising finding that synthetic, non-natural modified E2 proteins (“E2 majority”, “E2 majority R9” and “E2 majority w/o”) are recognized by sera obtained from patients infected with various HCV genotypes and by hyperimmune sera from mice immunized with various forms of E2. Moreover, these synthetic proteins can elicit a robust immune response that is capable of neutralizing HCV infection in an animal model. These results suggest that the unique structure of the synthetic E2 proteins can elicit an immune response towards various forms of E2 and therefore make it an ideal candidate for a vaccine.

### Materials and Methods

All general materials were purchased from Sigma (Israel). Mammalian E2 CHO was purchased from Austral Biologicals (CA).

All methods of expression and purification described below refer to all types of E2 disclosed in the invention (E2 majority, E2 majority R9 and E2 majority w/o) as well as to natural E2.

Generation of Synthetic HCVE2 cDNA 's

1. E2-Majority R9

This construct was synthesized by recursive PCR by using 2 separate PCR reactions, #'s 1 and 2. In PCR reaction # 1, the following 5 sense and 5 anti-sense primers were mixed together in a single tube:

Primer Data was Deleted

In this first PCR, all sense (no's 1-5) and anti-sense primers (no's 1-5) were mixed together at a stock concentration of 25 pmoles/μl. A 3 μl aliquot of the mixed primer was taken for PCR using the following program: 95° C., 3 min, 94° C. 30 sec. 58° C. 30 sec, 72° C. 1 min, 30 times followed by a 72° C. extension for 5 min with Bio-X-Act DNA Polymerase (Bioline, London, UK).

Following PCR # 1, a 1 μl aliquot was withdrawn and taken for PCR # 2 using external flanking, small oligonucleotides. In this reaction, the sense primer was 5′ cgc-gga-tcc-cag-acc-acc-gtg-gtt-g 3′ and the anti-sense primer was 5′ ccg-gaa-ttc-tta-tca-gtg-gtg-gtg-g 3′. The PCR conditions used were the same as for PCR # 1 with the exception that the program consisted of 20 cycles. PCR fragments were electrophoresed on 1% agarose gels, visualized with ethidium bromide under UV and purified prior to subsequent cloning with QIAquick gel extraction kit (Qiagen, Hilden, Germany). Purified fragments were enzymatically cleaved with BamH1 and EcoRI restriction enzymes and ligated into the Baculovirus expression plasmid pAcGP67B (Pharmingen, USA) previously digested with BamH1 and EcoRI.

2. E2-Majority w/o

PCR was performed on the previously described recombinant plasmid pGP67B harbouring E2 majority R9 cDNA using the following sense and anti-sense primers:

Primner Data was Deleted

The PCR conditions were the same as reaction #1 above. The resultant PCR fragment was gel purified, digested with BamH1 and EcoRI and ligated into the identically digested pAcGP67B plasmid.

3. E2 Majority

E2 majority was constructed using a combination of restriction enzyme digestion and recursive PCR.
a) E2 majority R9 was digested to completion with BamH1 and AscI restriction enzymes. The digest was electrophoresed on a 1% agarose gel and the larger 10 kb fragment was excised and purified.
b) A recursive PCR reaction was performed as in reaction # 1 using the following 19×˜50 mer oligonucleotides with 25 mer overhangs:

Primer Data was Deleted

The resultant ˜400 bp fragment was electrophoresed on a 1% agarose gel, purified, digested with BamH1/AscI and re-purified. This fragment was thereafter ligated into the digested E2 majority R9 plasmid generated previously (section (a) above). Following transformation into E. coli competent cells, bacterial colonies were grown, plasmid DNA isolated and submitted for DNA sequencing. Clones that matched the predicted DNA sequence were further grown and plasmid DNA extracted to generate the recombinant plasmid pAcGP67B E2 majority.

Baculovirus Cell Lines:

Adherent SF9 (Spodoptera frugiperda) cells were maintained in Grace's Insect Media (Biological Industries, Beit Haemek, Israel) supplemented with yeastolate, lactalbumin hydrolysate, 10% fetal calf serum, and 50 μg/ml gentamycin (TNM-FH media). These cells were used only for protocols involving transfection, end point dilution analysis (EPDA) and generation of high titer recombinant viruses. High-Five cells (Trichoplusia ni) were maintained as adherent or shaker cultures and grown in serum-free media (Insect Xpress, BioWhittaker, MD; Ex-Cell 405 media, JRH Biosciences, Andover UK). These cells were used for protein expression studies. All cells were maintained at 27° C. in a refrigerated incubator (VELP Scientific).

Generation of Recombinant Baculoviruses:

All recombinant Baculoviruses were generated using the linearized BaculoGold kit (Pharmingen, San Diego). EPDA and viral amplification procedures to generate high titer recombinant viral titer stocks (2×10⁸ pfU/ml) were performed according to Pharmingen's Instruction Manual.

Expression and Purification of Baculovirus E2 Majority Proteins:

Adaptation and Infection of High-Five Cells

Prior to protein expression studies, High-Five cells were adapted to shaker flasks. In brief, adherent cells (10⁶ cells per ml) from flasks (Corning Costar, Mass.) were transferred to 1 l Erlenmeyer Polycarbonate Flasks (Corning, Mass.) and shaken at 150 rpm for 24 hr in 500 ml serum-free media. Flasks were thereafter removed, placed in a tissue culture hood and tilted for 5 min to separate cell aggregates from non-aggregated cells. Homogenous, single cells were removed, transferred to new Erlenmeyer flasks, seeded at a density of 1×10⁶ cells per ml and infected with the panel of E2 majority recombinant Baculoviruses at a multiplicity of infection (MOI)=3. Three days following infection, shaker cultures were harvested, supernatants collected following centrifugation, sterile filtered and stored at 4° C. or −70° C.

RT-PCR on Baculovirus E2 Majority Infected High-Five Cells

To verify that expression and purification of E2 majority proteins was derived from its specific recombinant baculovirus, during some expression experiments 5×10⁶ cells were withdrawn and taken for RNA isolation using Tri-Reagent BD (Molecular Research Center, OH). An aliquot of RNA was taken for reverse transcription (RT)-primed using oligo-dT (Promega, WI) and catalyzed with AMV and MLV reverse transcriptases (Promega, WI). cDNA was taken for PCR using pAcGP67B sense and anti-sense primers (see above). The PCR program was 3 min at 94° C. (once), 1 min at 94° C., 1 min at 58° C., 1.5 min at 72° C. (33 cycles) followed by a final elongation step of 5 min at 72° C. PCR fragments were gel purified and submitted for direct DNA sequencing using pAcGP67B sense and anti-sense primers (see above).

Concentration of Infected Baculovirus E2 Majority Supernatants:

Various volumes of Baculovirus E2 majority supernatants (250 ml to 7 l) were concentrated ˜10 fold using Vivaflow 50, 200 (Vivascience, UK) or Pellicon-XL 50 (Millipore, MA) regenerated cellulose concentrator units. Concentrates were dialyzed against either PBS or native lysis buffer (NLB, pH=8) consisting of 50 mM NaHPO₄, 300 mM NaCl, 10 mM Imidazole overnight at 4° C. To the material following dialysis against NLB only, a 1/20 dilution of 10% Triton-X-100 in NLB was added. All dialyzed material was thereafter sterile filtered and taken for purification.

FPLC Purification of Baculovirus E2 Majority Supernatants:

Purification by Nickel (Ni-NTA) Agarose Columns:

Columns ranging from 4.5 to 30 ml Nickel-NTA Agarose (Qiagen, Hilden) were prepared in Pharmacia C columns. Columns were connected to an AKTA Explorer (Pharmacia, NJ) and washed with three column volumes of NLB at a flow rate of 3 ml per min. E2 majority supernatants were loaded at a rate of 1-2 ml per min and columns washed with 5 column volumes of NLB containing 20 mM Imidazole at 3 ml per min. E2 majority was eluted with 5 column volumes of NLB containing 300 mM Imidazole at a flow rate of 3 ml per min. Optical density of eluted fractions was measured at 280 nm, pooled and dialyzed extensively against PBS at 4° C.

Purification by Streptavidin-Sepharose Conjugated anti-E2 mAb 18 Columns:

Twenty-five mg, purified mab 18 (see below) was conjugated to 1.25 mg biotin (Pierce, Rockville, Ill.) and extensively dialyzed against PBS. Biotinylated mAb 18 (22 mg) was conjugated to 10 ml Streptavidin Sepharose High Performance (Pharmacia, NJ) for 30 min at room temperature by gentle agitation followed by loading onto an HR 10/10-column (Pharmacia, NJ). Bound biotinylated mAb 18 was verified by spectrophotometric determination (A₂₈₀ nm) of flow through material and columns were washed with PBS. Concentrated E2 supernatants previously dialyzed against PBS were loaded onto columns at a rate of 2 ml per min. Following a 5 column volume wash with PBS, bound material was eluted with 1 ml fractions of 0.1 M glycine (pH=3) and immediately neutralized with 50 μl 1 M Tris-base (pH=9). Optical density of eluted fractions was measured at 280 nm, pooled and dialyzed extensively against PBS at 4° C.

Determination of Protein Concentration:

Concentrations of purified proteins were determined by Bradford assay (Bio-Rad, CA).

Immunoblot Analysis:

Crude or purified protein samples (100-200 ng) under reducing (5 min heating at 95° C. in the presence of 360 mM β-mercaptoethanol) or non-reducing conditions (10 min at 37° C. without β-mercaptoethanol) supplemented with LDS-loading buffer were loaded onto 4-12% NuPAGE gels and electrophoresed in MES running buffer (Novex, San Diego). Proteins were electroblotted to nitrocellulose membranes by wet transfer using an XCell II Blot module (Novex, San Diego) and blocked overnight at 4° C. in blocking buffer (PBS-0.04% Tween-0.3% milk protein). Blots were incubated in fresh blocking buffer following the addition of penta-His (Qiagen, Hilden), mouse or human anti-E2 mAbs at 0.02-2 μg/ml for 3 hrs at room temperature. An identical protocol was performed using HCV patients' sera at various dilutions. Following three separate 5 min washes in block buffer, blots were incubated with either peroxidase conjugated goat anti-mouse (1:10,000) or goat anti-human IgG (1:20,000; Zymed Incorporation, South San Francisco) and taken for enhanced chemiluminescence (ECL).

Generation of Mouse Anti-E2 mAbs:

Immunization:

Anti-E2 mAb 18:

BALB/C (5 weeks old) were immunized with 10 μg E2 in Complete Freund's Adjuvant (Difco Laboratories, Detroit, Mich.) 1:1 volume via footpad. Mice were boosted twice every 2 weeks with 5 μg E2 in Incomplete Freund's adjuvant (Difco Labroatories, Detroit, Mich.) 1:1 volume via footpad. Mice were boosted with 1 μg E2 (i.v.) 3 days prior to harvesting of spleen for subsequent fusion.

Hyperimmune Sera and Anti-E2 Majority R9 mab 21:

BALB-C mice (5 weeks old) were immunized with 10 μg E2 or E2 majority R9 with 100 μg phosphorothioate containing CpG (5′ tcc-atg-acg-ttc-ctg-acg-tt 3′; Genset, France) and 25 μl of 2% Alum (Sigma, MO). This antigen mixture was vortexed and placed on ice for 30 min prior to i.p. immunization. Three days before spleens were harvested for fusion, mice were further immunized with 2 μg antigen i.v.

Fusion of Mice Spleen:

Spleen cells were mixed with human-mouse heteromyeloma HMMA2.11TG/0 at a 3:1 ratio. Fusion was performed with 50% (w/v) PEG 1500 (Boehringer Mannheim GmbH, Mannheim, Germany) and fused cells were seeded at a concentration of 30,000 cells per well in 96-well U-bottom microtiter plates (Nunc. Inc) in complete RPMI medium containing hypoxanthine, aminopterin and thymidine (HAT) supplement (1×) (Biological Industries, Beit Haemek, Israel). Cells were fed with fresh HAT medium 1 week later. Two weeks following fusion, supernatants were harvested for ELISA against the respective immunogens for the presence of specific antibodies. Medium was replenished with fresh hypoxanthine, thymidine (HT)-containing medium. Hybridoma cultures secreting specific anti-E2 or anti-E2 majority R9 mAbs were cloned by limiting dilution at 0.5 cell/well in 96 U-bottom microtiter plates.

Ascites Preparation of Clones 18 and 21:

BALB-C mice were injected i.p. with 500 μl Pristane (Sigma, MO). Ten days later, 5×10⁶ of specific mouse monoclonal antibodies were injected i.p. Three to four weeks later, a peritoneal lavage was performed and ascites fluid removed.

Purification of Ascites Fluid:

Ascites fluid was diluted 1:1 with PBS and loaded onto a 5 ml Hi-trap protein G column (Pharmacia, NJ) at 5 ml per min. Following washing with 40 ml PBS, bound mAb was eluted with 30 ml 0.1 M glycine (H=2.7) and dialyzed extensively against PBS. MAbs were stored at a concentration of 1 mg/ml at −20° C.

Elisa:

MaxiSorp ELISA plates (Nunc, Inc) were coated with 2 μg/ml E2 antigens in PBS (50 μl per well) overnight at 4° C. Following blocking with 200 μl PBS/1% BSA per well for 2 hr at 37° C., serial dilutions of mice sera (1:100-1:200,000) or purified mAb (1 μg/ml) was added to wells for 2 hr at 37° C. Following washing with PBS/0.04% Tween-20, a 1:10,000 dilution of peroxidase-conjugated goat anti-mouse IgG (H+L) was added for 1 hr at 37° C. followed by colorimetry at 450 nm using 3,3′,5,5′-tetramethyl-benzidine dihydrochloride (TMB, Sigma, MO) as the substrate.

### Example 1

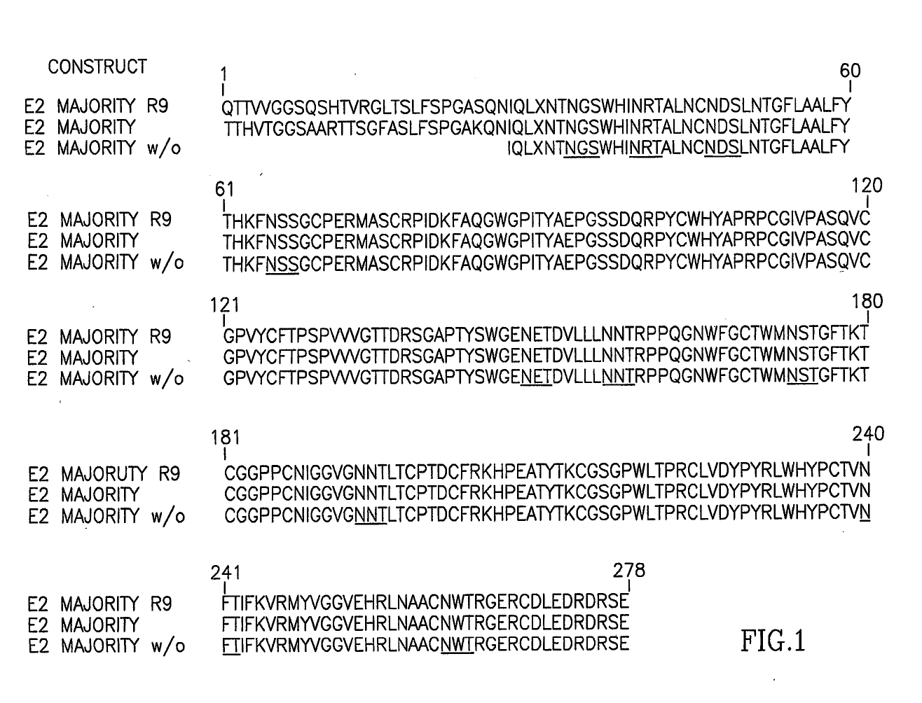

E2 majority was constructed by comparing the amino acid sequences of 77 different E2 proteins (from Genebank deposited sequences). The most frequently occurring amino acid in each position was selected and the construct was synthesized accordingly. E2 majority w/o is a truncated version of E2 majority lacking the first 27 amino acids comprising HVR1. E2 majority R9 contains in the HVR1 region a previously identified HVR1 mimotope termed R9 (27 amino acids long). E2 majority, E2 majority w/o and E2 majority R9 are otherwise identical (FIG. 1; Sequence listing: SEQ ID NO. 1 describes E2 majority, SEQ ID NO. 2 describes E2 majority w/o and SEQ ID NO. 3 describes E2 majority R9). The amino acid in position 31 (designated X) can be either phenylalanine (F) or isoleucine (I).

The synthetic constructs were generated by a recursive PCR methodology. Using a shotgun approach, respective primers (˜100 mer) with 20 mer 5′ and 3′ overhangs that encompassed the entire coding majority sequences were mixed and subjected to PCR (see methodology). To generate the final PCR product for cloning into pAcGP67B, smaller 5′ and 3′ primers were designed that contained 5′ BamHI and 3′ EcoRI coding sites. The 3′ primer harbored a histidine tag allowing purification.

High-Five cells were grown as shaking cultures and infected with a recombinant E2 (majority, majority R9 or majority w/o) baculovirus at a multiplicity of infection (MOI) of 3. Supernatant samples were withdrawn over a subsequent 72 hr time period and probed on protein gels with either anti-His or anti-E2 mAbs. Imnunoreactive E2 (Mw=50 kD) was observed, albeit very faintly, as early as 8 hr after infection which increased up to 72 hr post-infection. This time point was subsequently chosen for collection of supernatants.

In order to obtain pure E2 majority proteins the supernatants containing the proteins can be concentrated, dialyzed and loaded onto either Nickel-NTA agarose columns or anti-E2 mAb affinity columns as described (see methodology). Identical flow-through (FT), wash and elution (Eln) fractions can be loaded onto separate SDS-PAGE and processed for anti-His mAb reactivity and Coomassie Blue stain respectively to test for the efficiency of purification.

### Example 2

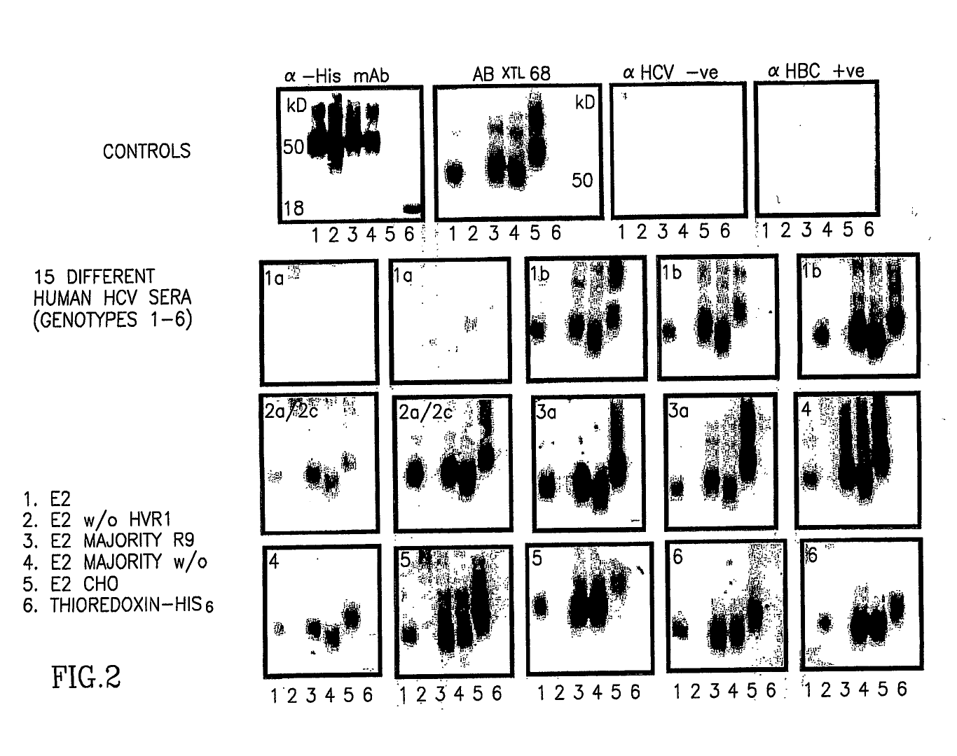

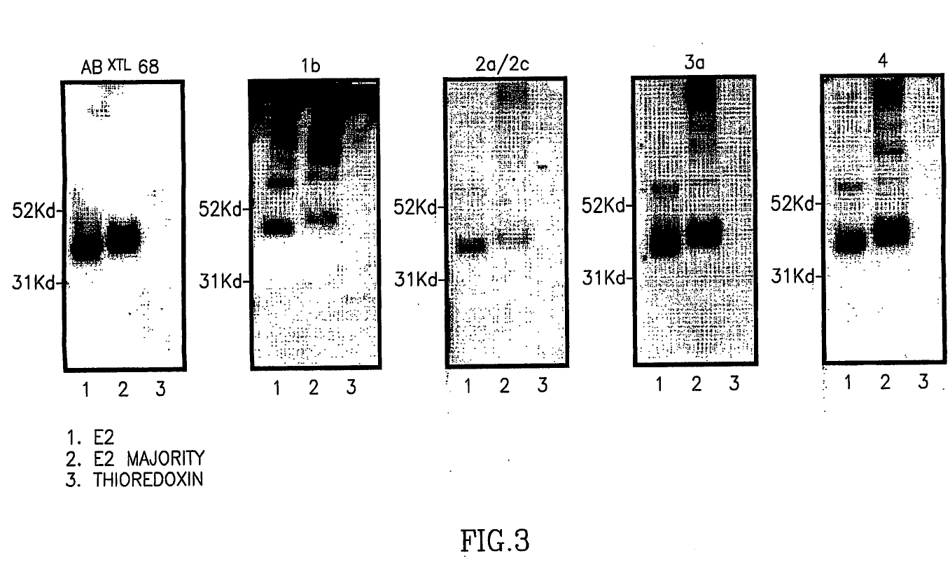

To investigate the functionality of the synthetic majority proteins, non-reducing immunoblots were performed and probed with sera from various HCV genotypes (FIGS. 2 and 3). In the Western blot shown in FIG. 2 various baculovirus expressed E2 preparations, equivalent amounts (100 ng) of E2 CHO and thioredoxin were run on the gel. Using an appropriate sera dilution range (1:2,500-1:10,000), robust signals were observed for E2 majority R9 and E2 majority w/o (lanes 3 and 4) with most sera types tested. Other E2 proteins also showed reactivity with the different sera. The E2 w/o HVR1 preparation in lane 2 lacks 31 amino acids at the N-terminus of E2 i.e. HVR1+additional 4 amino acids and is the only E2 preparation with which none of the sera reacted. This implies indirectly that these 4 amino acids are very important for conserving the correct conformation of E2 that allows antibody recognition. AB^{XTL}68 (0.5 μg/ml), a fully human anti-E2 mAb served as a positive control whereas α-HCV negative and α-HBC positive sera (both at 1:2,500 dilution) served as negative controls.

In a separate experiment, non-reducing immunoblots were performed and probed with sera from HCV genotypes 1-4 (FIG. 3). E2 majority was recognized by all sera types and by AB^{XTL} 68.

### Example 3

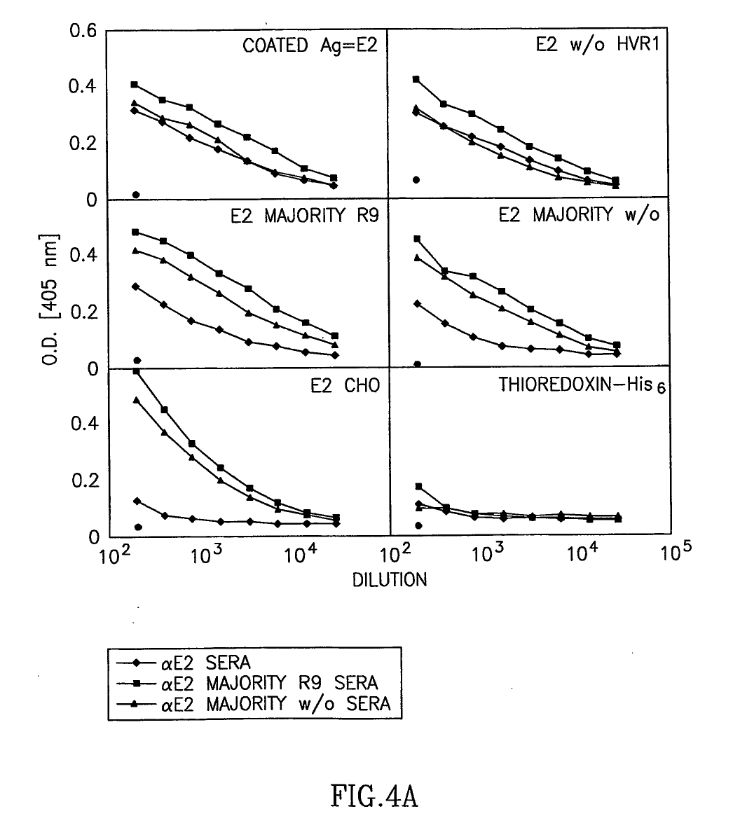

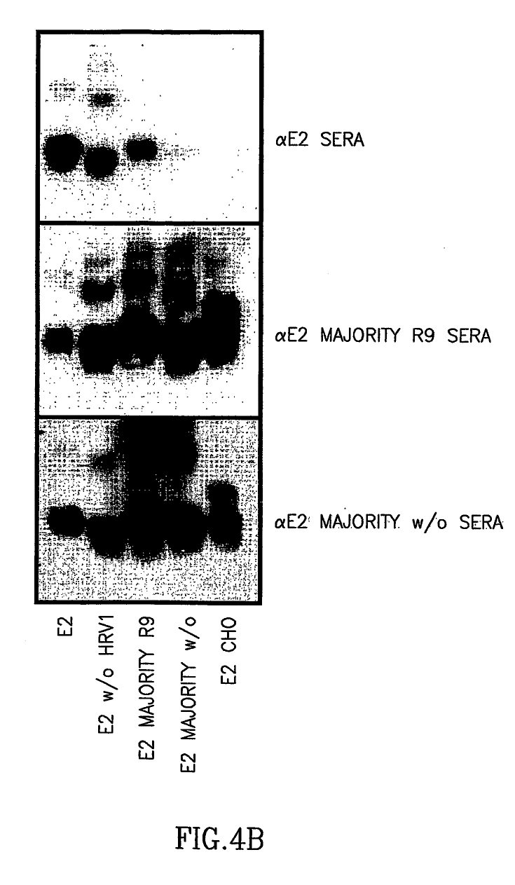

Mice were immunized with natural E2, E2 majority R9, and E2 majority w/o. Hyperimmune sera was obtained from the mice and was assessed for reactivity against immobilized antigens by ELISA. Hyperimmune sera from mice immunized with E2 majority R9, and E2 majority w/o showed highest reactivity towards all 4 baculovirus produced E2 proteins (FIG. 4A). Interestingly, pronounced reactivity was also observed against mammalian E2 (E2 CHO), an effect not observed with hyperimmune sera from mice immunized with natural E2. No reactivity of hyperimmune sera was observed towards thioredoxin (FIG. 4A). In support of these data, on reducing SDS-PAGE, hyperimmune sera from mice immunized with E2 majority R9 or E2 majority w/o showed a specific, broad reactivity to all baculovirus-expressed E2 antigens as well as E2 CHO (FIG. 4B). Conversely, hyperimmune sera from mice immunized with natural E2 exhibited a restricted pattern of reactivity (FIG. 4B). These results indicate that the majority proteins are capable of inducing an immune response directed against a wide range of E2 proteins in contrast to the natural E2, which is capable of raising a specific response directed only towards the injected antigen. The broad-spectrum immune response indicates that the majority proteins may have an advantage over the natural E2 as vaccines.

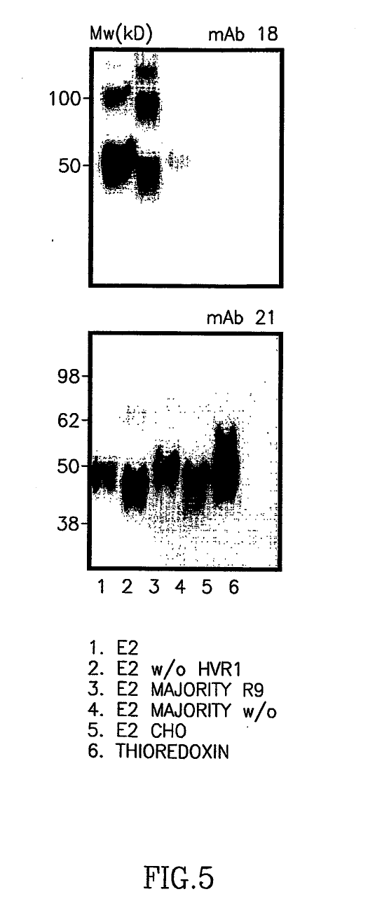

Similarly, monoclonal antibodies (mkbs) generated against the E2 majority R9 react with several types of E2, while mAbs raised against the natural E2 are more restricted in their spectrum of recognition. Following immunization of mice with E2 or E2 majority R9 and subsequent fusion of spleens, two different mAbs were generated. The binding characteristics of these mAbs was tested on reducing SDS-PAGE, α-E2 in Ab 18 recognized only the natural E2s (with or w/o HVR1) while the anti-E2 majority R9 mAb 21 recognized all baculovirus-expressed E2 antigens as well as E2 CHO (FIG. 5).

### Example 4

The biological activity of sera directed against various E2 preparations was characterized using the following HCV-Trimera animal model: a mouse was treated so as to allow the stable engraftment of human liver fragments. The treatment included intensive irradiation followed by transplantation of said (severe combined immuno deficient) mice bone marrow. Viral infection of human liver fragments was performed ex vivo using HCV positive human serum (U.S. Pat. No. 5,849,987).

0.5 ml samples of human sera containing 7.5×10⁵ HCV-RNA copies/ml with no detectable anti E2 IgG were pre-incubated for 3 h at room temperature with various anti sera according to the following:
IgG fraction from mice immunized with natural E2 (300 μg);
IgG fraction from mice immunized with E2 majority R9 (300 μg);
IgG fraction from mice immunized with E2 majority w/o (300 μg);
Pre-immune sera served as a negative control.

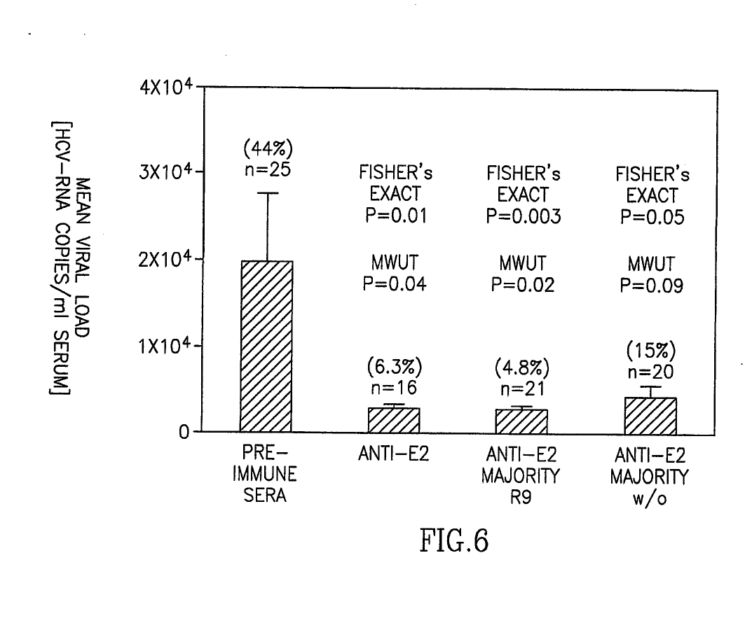

The pre-incubated sera were subsequently used to infect normal human liver fragments ex vivo. Following infection, the liver fragments were transplanted in mice and HCV-RNA was determined in sera 19 days later. FIG. 6 shows the effect of the various anti E2 antibody preparations in inhibiting liver infection by HCV, as demonstrated by both the mean viral load and the percentage of HCV-RNA positive mice. Sera generated against natural E2, E2-majority R9 and E2-majority w/o reduced significantly the mean viral load and the percentage of infected animals.

1. A purified and isolated E2 protein having a sequence selected from the group consisting of SEQ ID NO: 1-3 or a variant thereof.

2. A purified and isolated E2 protein according to claim 1 further linked to another polypeptide.

3. A composition consisting essentially of an E2 protein according to claim 1 and an appropriate carrier.

4. A purified and isolated nucleic acid encoding an E2 protein according to claim 1.

5. A purified and isolated nucleic acid encoding an E2 protein according to claim 1 having a nucleotide sequence selected from the group consisting of SEQ ID NO: 4-6.

6. An expression vector comprising a nucleic acid according to claim 1.

7. A host organism transformed or transfected with an expression vector according to claim 6.

8. An E2 protein produced by the host organism of claim 7.

9. A method of preventing hepatitis C comprising administering to an individual the composition of claim 3 in an amount capable of stimulating the production of a sufficient level of protective antibodies.

10. A method for treating hepatitis C comprising administering to an HCV carrier the composition of claim 3 in an amount capable of eliciting an immune response against HCV.

11. A vaccine for immunizing an individual against hepatitis C consisting essentially of an E2 protein according to claim 1 in a pharmaceutically acceptable carrier.

12. A composition comprising an expression vector according to claim 6.

13. A method of preventing hepatitis C comprising administering to an individual the composition of claim 12 in an amount capable of stimulating the production of a sufficient level of protective antibodies.

14. A method for treating hepatitis C comprising administering to an HCV carrier the composition of claim 12 in an amount capable of eliciting an immune response against HCV.

15. A vaccine for immunizing an individual against hepatitis C consisting essentially of an expression vector according to claim 6 in a pharmaceutically acceptable carrier.

16. Anti E2 antibodies having specific binding affinity for an E2 amino acid sequence selected from the group consisting of SEQ ID NO: 1-3.

17. A method of preventing hepatitis C comprising administering the antibodies of claim 16 to an individual in an amount effective to protect said individual from challenge with HCV.

18. Use of the E2 protein according to claim 1 for the manufacture of an HCV vaccine composition.

19. Use of the E2 protein according to claim 1 for inducing immunity against HCV in chronic HCV carriers.

20. Use of the E2 protein according to claim 19 for inducing immunity against HCV in chronic HCV carriers prior to, simultaneously to or after any other therapy.

21. Use of the E2 protein according to claim 1 for inducing immunity against HCV in HCV-infected individuals prior to or after liver transplantation, or after presumed infection.

22. Use of the E2 protein according to claim 1 or prophylactically inducing immunity against HCV.

23. The E2 protein or the composition according to claim 1 for use as an HCV vaccine.

24. The E2 protein or the composition according to claim 1 for inducing immunity against HCV in chronic HCV carriers.

25. The E2 protein or the composition according to claim 24 for inducing immunity against HCV in chronic HCV carriers prior to, simultaneously to or after any other therapy.

26. The E2 protein or the composition according to claim 1 for inducing immunity against HCV in HCV-infected individuals prior to or after liver transplantation, or after presumed infection.

27. The E2 protein or the composition according to claim 1 for prophylactically inducing immunity against HCV.

28. Use of the specific antibodies according to claim 16 to treat or prevent HCV infection.

29. Kit for detecting HCV antibodies present in a biological sample, comprising the E2 protein according to claim 1 in a suitable container.

30. Immunoassay for detecting HCV antibody, which immunoassay comprises:
(1) Providing the E2 protein according to claim 1,
(2) Incubating a biological sample with said E2 protein under conditions that allow formation of antibody-antigen complex,
(3) Determining whether said antibody-antigen complex comprising said E2 protein is formed.


SEQUENCE LISTING (9 sequences)

SEQ ID NO: 1 - protein, 278 aa, Artificial Sequence
  Description of Artificial Sequence Synthetic Construct E2 Majority
TTHVTGGSAARTTSGFASLFSPGAKQNIQLXNTNGSWHINRTALNCNDSLNTGFLAALFYTHKFNSSGCPERMASCRPIDKFAQGWGPITYAEPGSSDQRPYCWHYAPRPCGIVPASQVCGPVYCFTPSPVVVGTTDRSGAPTYSWGENETDVLLLNNTRPPQGNWFGCTWMNSTGFTKTCGGPPCNIGGVGNNTLTCPTDCFRKHPEATYTKCGSGPWLTPRCLVDYPYRLWHYPCTVNFTIFKVRMYVGGVEHRLNAACNWTRGERCDLEDRDRSE

SEQ ID NO: 2 - protein, 251 aa, Artificial Sequence
  Description of Artificial Sequence Synthetic Construct E2 Majority w/o
IQLXNTNGSWHINRTALNCNDSLNTGFLAALFYTHKFNSSGCPERMASCRPIDKFAQGWGPITYAEPGSSDQRPYCWHYAPRPCGIVPASQVCGPVYCFTPSPVVVGTTDRSGAPTYSWGENETDVLLLNNTRPPQGNWFGCTWMNSTGFTKTCGGPPCNIGGVGNNTLTCPTDCFRKHPEATYTKCGSGPWLTPRCLVDYPYRLWHYPCTVNFTIFKVRMYVGGVEHRLNAACNWTRGERCDLEDRDRSE

SEQ ID NO: 3 - protein, 278 aa, Artificial Sequence
  Description of Artificial Sequence Synthetic Construct E2 Majority R9
QTTVVGGSQSHTVRGLTSLFSPGASQNIQLXNTNGSWHINRTALNCNDSLNTGFLAALFYTHKFNSSGCPERMASCRPIDKFAQGWGPITYAEPGSSDQRPYCWHYAPRPCGIVPASQVCGPVYCFTPSPVVVGTTDRSGAPTYSWGENETDVLLLNNTRPPQGNWFGCTWMNSTGFTKTCGGPPCNIGGVGNNTLTCPTDCFRKHPEATYTKCGSGPWLTPRCLVDYPYRLWHYPCTVNFTIFKVRMYVGGVEHRLNAACNWTRGERCDLEDRDRSE

SEQ ID NO: 4 - DNA, 834 bp, Artificial Sequence
  Description of Artificial Sequence Synthetic Construct E2 Majority
accacccacgtgaccggcggcagcgccgcccgcaccaccagcggcttcgccagcctgttcagccccggcgccaagcagaacattcagctgtttaacaccaacggtagttggcacattaaccgtacggccctgaactgcaacgatagcctgaatacggggtttctggccgccttgttctatacgcataagttcaactctagtggttgcccagaacgtatggcgagctgccgtccgattgataaattcgcgcagggctggggcccgattacctatgcggaaccgggcagctctgatcagcgtccgtattgctggcattacgcgccacgtccgtgtggcattgtgccggcgagccaagtttgtggcccggtgtattgcttcaccccgagcccggtggtggtgggtacgacggaccgcagcggcgcgccgacgtacagctggggcgaaaacgaaaccgacgttctgttactcaataatacgcgtccaccacaaggcaactggttcggctgcacctggatgaactctacgggtttcactaaaacctgcggcggcccgccgtgcaacattggcggtgttggtaacaacaccctgacctgcccgaccgattgcttccgtaaacacccggaagcgacctataccaaatgcggctctggcccgtggctgaccccgcgttgcctggtggattatccgtatcgtctgtggcactatccgtgcaccgtgaacttcaccattttcaaagttcgcatgtatgttggtggtgttgagcaccgtctcaacgctgcatgcaactggacccgtggcgaacgttgcgatctggaagatcgtgatcgtagcgaa

SEQ ID NO: 5 - DNA, 834 bp, Artificial Sequence
  Description of Artificial Sequence Synthetic Construct E2 Majority w/o
cagaccaccgtggttggcggtagccagtctcacaccgtgcgtggcctgaccagcctgttcagcccgggtgcctctcaaaacattcagctgtttaacaccaacggtagttggcacattaaccgtacggccctgaactgcaacgatagcctgaatacggggtttctggccgccttgttctatacgcataagttcaactctagtggttgcccagaacgtatggcgagctgccgtccgattgataaattcgcgcagggctggggcccgattacctatgcggaaccgggcagctctgatcagcgtccgtattgctggcattacgcgccacgtccgtgtggcattgtgccggcgagccaagtttgtggcccggtgtattgcttcaccccgagcccggtggtggtgggtacgacggaccgcagcggcgcgccgacgtacagctggggcgaaaacgaaaccgacgttctgttactcaataatacgcgtccaccacaaggcaactggttcggctgcacctggatgaactctacgggtttcactaaaacctgcggcggcccgccgtgcaacattggcggtgttggtaacaacaccctgacctgcccgaccgattgcttccgtaaacacccggaagcgacctataccaaatgcggctctggcccgtggctgaccccgcgttgcctggtggattatccgtatcgtctgtggcactatccgtgcaccgtgaacttcaccattttcaaagttcgcatgtatgttggtggtgttgagcaccgtctcaacgctgcatgcaactggacccgtggcgaacgttgcgatctggaagatcgtgatcgtagcgaa

SEQ ID NO: 6 - DNA, 834 bp, Artificial Sequence; identical to SEQ ID NO: 5
  Description of Artificial Sequence Synthetic Construct E2 Majority R9

SEQ ID NO: 7 - DNA, 25 bp, Artificial Sequence
  Description of Artificial Sequence Synthetic Primer
cgcggatcccagaccaccgtggttg

SEQ ID NO: 8 - DNA, 25 bp, Artificial Sequence
  Description of Artificial Sequence Synthetic Primer
ccggaattcttatcagtggtggtgg

SEQ ID NO: 9 - DNA, 20 bp, Artificial Sequence
  Description of Artificial Sequence Synthetic Primer
tccatgacgttcctgacgtt